(19) United States
(12) Patent Application Publication
(10) Pub. No.: US 2010/0173366 A1
(43) Pub. Date: Jul. 8, 2010

(54) Polypeptides Having L-Arabinose Isomerase Activity Exhibiting Minimum Dependence on Metal Ions for Its Activity and for Thermostability and Nucleic Acids Encoding the Same

(75) Inventors: Moez Rhimi, Sidi Bouzid (TN); Hichem Chouayekh, Stax (TN); Mamdouh Ben Ali, Sfax (TN); Belgacem Naili, Gabes (TN); Sonia Jemli, Sfax (TN); Samir Bejar, Sfax (TN)

Correspondence Address: LADAS & PARRY LLP, 26 WEST 61ST STREET, NEW YORK, NY, 10023, US

(73) Assignee: CENTRE OF BIOTECHNOLOGY OF SAFX, SFAX-Tunisia (TU)

(21) Appl. No.: 11/794,510

(22) PCT Filed: Jun. 28, 2005
(86) PCT No.: PCT/TN2005/000005

§ 371 Date: Aug. 29, 2007

(30) Foreign Application Priority Data
Dec. 29, 2004 (TN) SN04264

(51) Int. Cl. C12P 19/24; C12N 15/61; C12N 9/90; C12N 15/63; C12N 1/00

(52) U.S. Cl. 435/94; 536/23.2; 435/233; 435/320.1; 435/243

(57) ABSTRACT

The invention concerns identification of a gene encoding a novel L-arabinose isomerase of the Bacillus stearothernivphilus strain US 100 (L-AI US 100), a L-arabinose isomerase expressed from said gene, recombinant vectors harbouring said gene, microorganisms transformed with said vector, a protocol for preparing and purifying said recombinant protein, biochemical and kinetic characterization of said recombinant enzyme and a method for bioconversion of a D-galactose solution into a solution rich in D-tagatose using said polypeptide. This novel protein has original characteristics, in particular its independence from metal ions for its activity and its low need for such ions for its thermostability, as well as its potential for isomerizing D-galactose into D-tagatose with great efficacy of about 48% after 7 hours at 70° C.

### INVENTION FIELD

This patent concerns the production and exploitation of enzymes having industrial interest in agro-alimentary or others application domains. Particularly, this invention concerns an L-arabinose isomerase having interesting physico-chemical properties which could be used at industrial scale.

The purpose of this invention is the cloning and sequencing of gene encoding a new L-arabinose isomerase, as well the sequence analysis of amino acids sequence corresponding to the enzyme. It concerns also the characterization and the use of an L-arabinose isomerase having original characteristics.

The L-arabinose isomerases are also known as D-galactose isomerases, in fact they are able to bio-transform the D-galactose into D-tagatose. In fact, this property has a potential industrial interest, since it is used for D-tagatose production.

The D-tagatose is a natural ketohexose isomer of D-fructose. It has a sweetness power similar to sucrose and a very low calorific energy. This ketose is used as a sweetener, or in association with other component having a high calorific and sweetener power in agro-alimentary industry. Regarding its anti-hyperglycemiant effect, the D-tagatose is important essentially for diabetic subjects. In pharmaceutical domain this sugar is employed for the conservation of organs in addition to its antibiofilm effect. However, the production of this sugar at industrial scale is limited by the high production costs. In order to surmount this limit, a new process was performed. This earlier process is based on the use of L-arabinose isomerase, which is able to transform the D-galactose into D-tagatose. In this context, many patents such as: USPA No: 20030022844; U.S. Pat. No. 6,057,135; USPA No: 20040058419 et USPA No: 20030175909 proved the feasibility of this procedure. In this bioconversion process, there are formation of a mixture of D-galactose and D-tagatose. In order to shift the equilibrium in favor of the D-tagatose, the isomerisation reaction should carry out at high temperatures. In other part, the L-arabinose isomerases are a metalloproteins which requires relatively high concentrations of metallic ions for their optimal activity. Indeed, these ions are not tolerable in final products and should be removed, requiring another step of purification which increases the process cost effectiveness. Consequently, it is of interest to screen newly thermoactive and thermostable L-arabinose isomerases having a feeble requirement of metallic ions. This will allow bioconversion at high temperature in presence of as low as possible metallic ions concentration.

The L-arabinose isomerase derived from the strain US100 which we describe in this report, have the originality to have a high optimal temperature (80° C.), high catalytic efficiency, independency towards metallic ions for its thermoactivity and a feeble requirement for its thermostability

### ABSTRACT OF THE INVENTION

In its specific form, this invention is characterized as following:

a) The screening of the L-arabinose isomerase activity of the Bacillus stearothermophilus (strain US100)

b) Cloning of the gene encoding this activity in an E coli strain.

c) Determination of the nucleotide sequence of the gene, deduction and analysis of the corresponding aa sequence encoding the enzyme.

d) The over-expression and purification of the recombinant L-arabinose isomerase of Bacillus stearothermophilus (strain US 100)

e) Biochemical and kinetic characterization of the recombinant L-arabinose isomerase of Bacillus stearothermophilus (strain US 100)

f) Use of the purified enzyme for the bioconversion of a D-galactose solution into rich D-tagatose solution

### BRIEF DESCRIPTION OF DRAWINGS

The characteristics of this present invention will be exposed by description listed below in conjunction with figures and tables, given as indication and not for restriction.

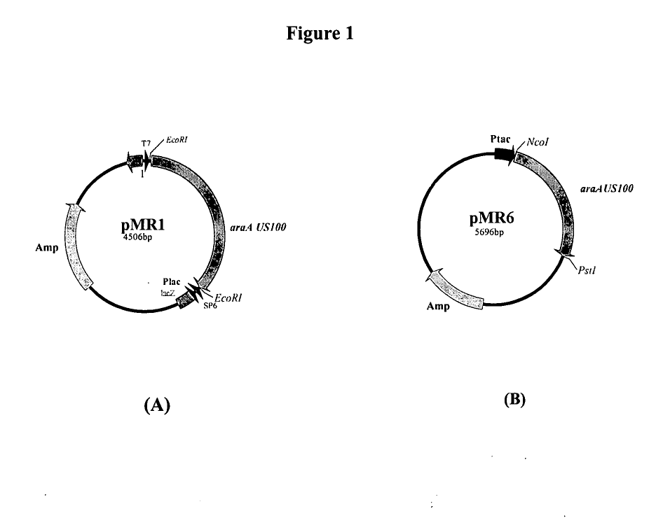

The FIG. 1 shows the restriction map of the expression vectors pMR1 (A) and pMR6 (B) containing the araA US100 gene of the L-AI US100, subject of this invention. Amp: gene encoding the ampicillin resistance, T7: promoter T7, SP6: promoter SP6.

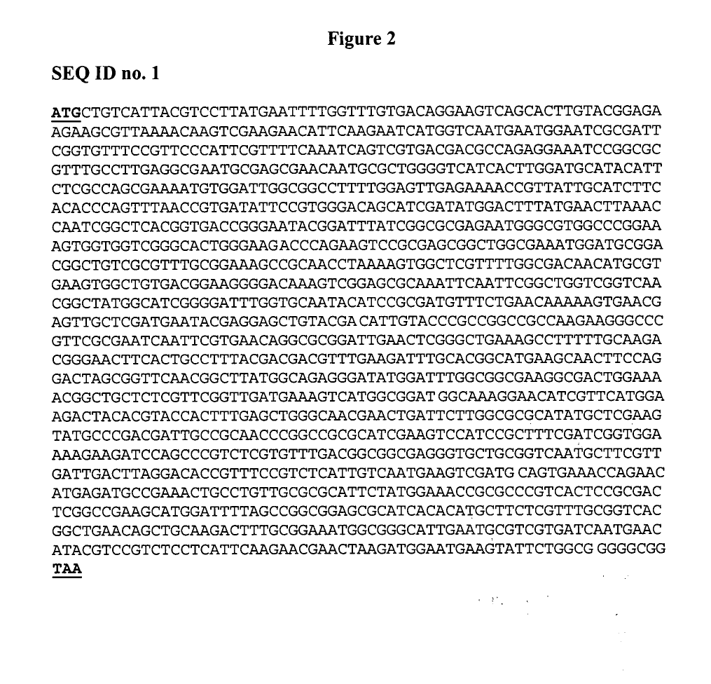

The FIG. 2 show the nucleotide sequence of the araA US 100 gene of the Bacillus stearothermophilus US100 encoding the L-arabinose isomerase subject of this invention. The ATG codon (translation start) and the TAA (stop) are represented in bold and underlined.

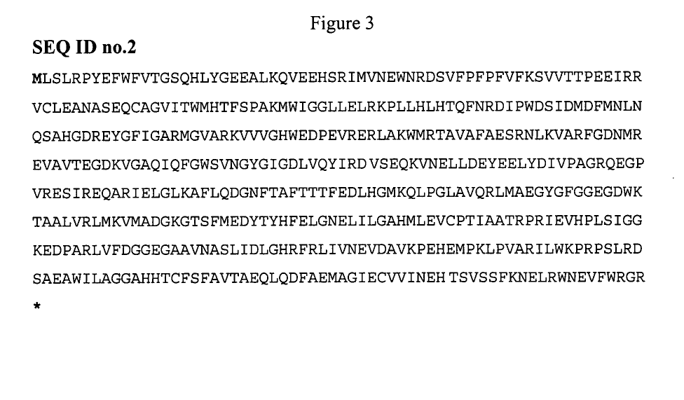

The FIG. 3 showed the amino acids sequence of the L-AI US 100 subject of this invention encoded by the araA US100 gene.

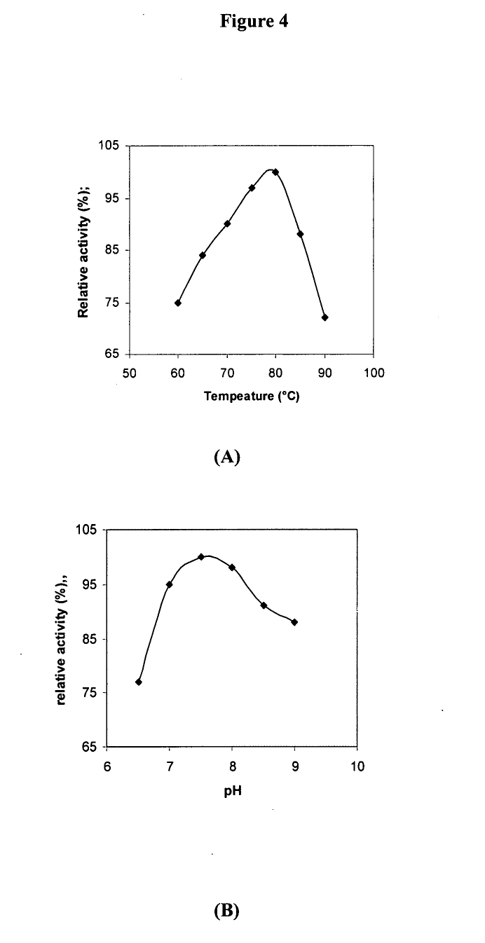

The FIG. 4 show the effect of temperature (A) and pH (B) on the activity of the L-arabinose isomerase, object of this invention using L-arabinose as substrate in presence of 0.2 mM Co²⁺ et 1 mM Mn²⁺. The activity determined at 100% corresponds to that measured under optimal conditions which correspond to 185 U/mg.

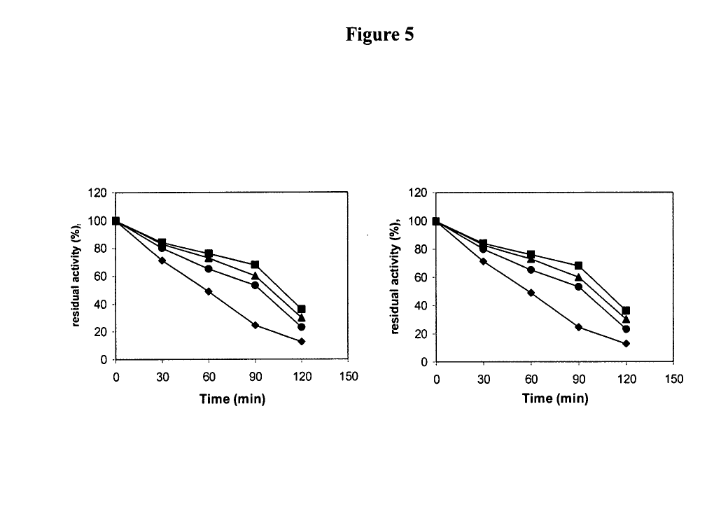

The FIG. 5 illustrates the effect of metallic ions concentration on the L-AI US100 thermostability; (A): effect of Co²⁺: 0 mM (♦), 0.1 mM (), 0.2 mM (▪), 0.4 mM (▴) and (B) effet du Mn²⁺: 0 mM (♦), 0.8 mM (), 1 mM (▪), 1.2 mM (▴). The activity determined at 100% corresponds to that measured under optimal conditions which correspond to 185 U/mg.

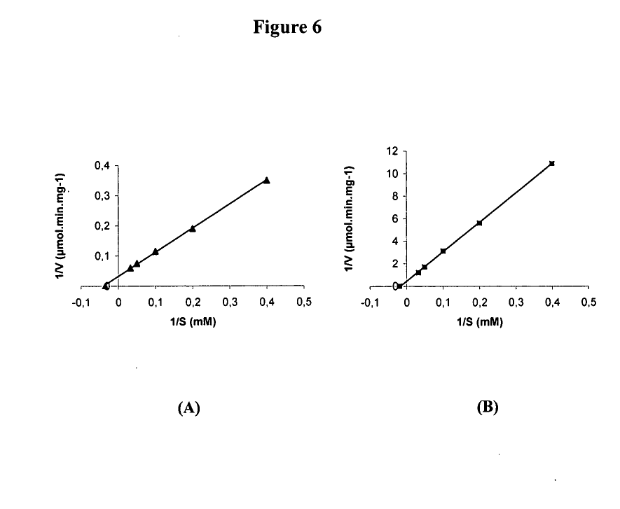

The FIG. 6 shows the Lineweaver and Burck graphic representation of the L-arabinose isomerase, object of this invention using the L-arabinose and the D-galactose as substrates.

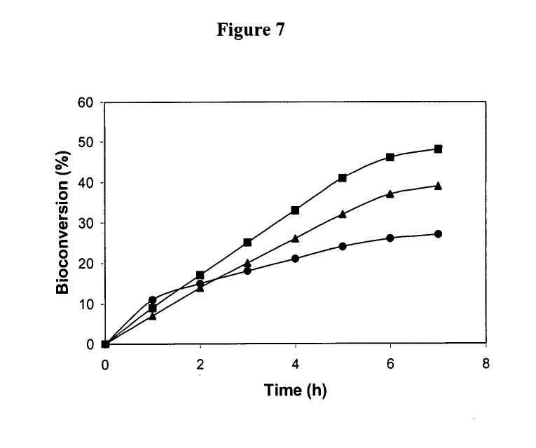

The FIG. 7 displays the D-galactose bioconversion rate to D-tagatose obtained using the purified L-arabinose isomerase object of this invention in presence of 0.2 mM Co²⁺ and 1 mM Mn²⁺ at different temperatures : 65° C. (▴), 70° C. (▪) et 75° C. ().

The Table 1 illustrates the effect of metallic ions on the L-AI US100 activity. (A) The activity determined at 100% corresponds to that measured under optimal conditions which correspond to 185 U/mg. (B) the ions Co²⁺ and Mn²⁺ are added with concentration evaluated to 0.1 mM.

The Table 2 shows catalytic parameters of the purified L-arabinose isomerase in comparison with others reported bacterial L-arabinose isomerases; nd: not determined.

### DETAILED DESCRIPTION OF THE INVENTION

The step (a) consists on the screening of many thermophiles strains for their ability to grow in a selective minimal medium containing L-arabinose as unique carbon source. The preliminary studies of the L-AI activities of these different strains prompted us to retain the Bacillus stearothermophilus (strain US 100) which was identified in a previous work, as an amylase producer strain (Tunsian patent No 17236 INNORPI TUNISIE, 2001; Mamdouh Ben Ali, Monia Mezghani and Samir Bejar. Enz. Microb. Technol. 24: 584-589, 1999).

The step (b) consists on the amplification of a polynucleotide encoding this activity by using the genomic DNA of the US 100 strain as template. The fragment of interest was cloned in the appropriate vector; the ligation product was used to transform the E. coli competent cells. Selection of recombinant clones was performed on MacConkey-L-arabinose with ampicillin.

The activity analysis carried out using the recombinant strains proved that they have an L-arabinose isomerase activiy, confirming the molecular cloning and the expression of this araA US100gene.

The step (c) consists on the determination of the nucleotide sequence of the araA US100 gene. This sequence is composed of 1491 pb encoding a polypeptide composed of 496 aa showing a significant homology with others L-AIs sequences previously reported in data bank.

The step (d) consists on over-expression and purification of the L-AI US 100. This recombinant strain was used for the enzyme purification. The established purification procedure consists on the heat treatment of the crude protein extract, ammonium sulphate precipitation and an anions exchange chromatography step (FPLC). The protein purity was checked by SDS-PAGE which displays a single band with 56 kDa. Under none denaturating conditions the protein has a molecular weight of nearly 225 kDa. The step (e) consists on the study of the biochemical characteristics of the recombinant enzyme, notably its profiles of pH, temperature and thermostability as well as its requirement on metallic ions. This study revealed an original property of the enzyme, which concern its independency towards metallic ions for its thermostability. In fact, this enzyme requires only 0.2 mM Co²⁺ and 1 mM Mn²⁺ for its maximal stability which contrast with the majority of L-AI previously described. The performed L-AI US100 kinetic studies allow the determination of the kinetic parameters: Kₘ, Vₘₐₓ, K_{cat} et Kₘ/K_{cat} for L-arabinose and D-galactose.

The step (f) consists on the study of the bioconversion of a D-galactose solution into rich D-tagatose solution using the purified L-AI US100. These experiments were done at different temperatures.

### Examples

Personifications of the present invention will be illustrated in examples given below, which should not be considered as limit of the capacities of the invention.

### Example 1

### Identification of Nucleotide Sequence of the L-arabinose Isomerase of Bacillus stearothermophilus (Strain US100) and its Heterologous Expressin in E. coli

In this example, are given data for indication and not for restriction, the identification of the nucleotide sequence of the L-arabinose isomerase of Bacillus stearothermophilus (strain US100) and its heterologous expression in E. coli.

The thermophile strain Bacillus stearothermophilus (strain US100) was grown in the appropriate medium at 55° C. The culture was used for the preparation of genomic DNA. Based on the sequence of the Bacillus setearothermophilus T6 arabinose operon (accession number: AAD45178) previously reported in the Genebank, we conceived two oligonucleotides: O1 ^{5′}GTGAACGGGGAGGAGCAATG^{3′} and O2 ^{5′}GAAATCTTACCGCCCCCGCC^{3′} in order to amplify the araA US 100 gene encoding the L-arabinose isomerase of Bacillus stearothermophilus (strain US 100).

A PCR fragment of approximately 1.5 kb was obtained using the two primers and the US 100 chromosomal DNA as the template. The fragment of interest, which would contain the araA US 100 gene, was purified, cloned in pGEMT-Easy Vector and introduced into E. coli HB101 strain. Several red clones carrying the araA US100 gene were obtained. One of them, containing a plasmid called pMR1, carrying the araA US 100 gene under the control of T7 promoter, was used for a liquid culture followed by the monitoring of the L-AI activity in the crude extract.

Determination of L-AI activity was performed using 50 μl of protein crude extract (HB101/pMR1 strain), 0.1 M MOPS (pH 7.5), 5 mM L-arabinose or D-galactose during 1 or 10 min at 80° C. The generated L-ribulose and D-tagatose was detected using cysteine carbazole method (Dische, Z., and. Borenfreund, E., J. Biol. Chem., 192:583-587, 1951). This shows that the HB101/pMR1 has a clearly detectable activity confirming the molecular cloning and expression of the araA US 100 gene.

### Example 2

### Sequencing of the araA US100 Gene and Analysis of the Corresponding aa Sequence

In this example the polynucleotide sequence determination, encoding the L-AI US100 activity and analysis of the corresponding aa sequence; is given for indication and not for restriction.

Using the pMR1 plasmid we have determined the polynucleotide (ara AUS100 gene) encoding the LAI US100 activity). The analysis of this sequence revealed the presence of a single Open Reading Frame of 1491 pb started with an ATG and ended with the TAA end codon (FIG. 2). The aa sequence deduced from the nucleotide sequence showed that L-AI US100 composed of 496 aa (FIG. 3). The analysis of this sequence reveals a high identities with those of other L-AIs attaining 98% (Table 1). In fact, the L-AI US100 have only 8 and 11 aa different from L-AIs derived from Thermus sp (accession number: AAO72082) and B. stearothermophilus T6 (accession number: AAD45718) respectively.

### Example 3

### Over-Expression and Purification of the L-AI US100

In this example, are given a data for indication and not for restriction the over-expression and purification of the L-AI US100

To over-express the L-AI US100, the corresponding gene was placed downstream of Ptac promoter of the ptrc99a vector generating the pMR6 plasmid (FIG. 1B). The determination of the L-AI US100 activities of the ER2566/pMR1 and HB101/pMR6 strains showed specific activities of 38.6 and 51 U/mg respectively. Hence, the efficient L-AI US100 expression was obtained with the Ptac-araA US100 construction (pMR6), which was retained for the L-AI US100 purification.

The enzyme was purified from the crude cell extract of an overnight HB101/pMR6 liquid culture. Taking advantage of the L-AI US100 thermostability, a heat treatment step in the presence of 0.2 mM Co²⁺ and 1 mM Mn²⁺ during 30 min at 70° C. followed by centrifugation (25000 rpm, 30 min), was introduced. This step removed the majority of thermolabile E. coli proteins. The heat treated crude protein extract was the subject of fractioned precipitation with ammonium sulfate followed by ion exchange liquid chromatography (UnoQ Column) step. Electrophoresis of the obtained protein preparation under reducing conditions (SDS-PAGE, Sodium Dodecyl Sulfate Polyacrylamid gel electrophoresis) revealed a single band at a molecular mass of about 56 kDa. Moreover, this preparation was a homogeneous enzyme with high purity as it exhibited a single band protein species of nearly 225 kDa on SDS-PAGE under non reducing conditions suggesting that the L-AI US100 is a holoenzyme composed of four identical subunits.

### Example 4

### Biochemical and Kinetic Studies of the L-arabinose Isomerase of the Strain US100

In this example are given data, for indication and not for restriction, concerning the biochemical and kinetic studies of the L-arabinose isomerase of the strain US100.

### A—Effect of Temperature and pH on the L-AI US100

The recombinant enzyme was purified as reported in the Example 2, the determination and measure of the activity was done as cited in the Example 1. The study of the activity at different temperature is shown in FIG. 4A. This study proved that the L-A1 US100 is active at different temperatures comprised between 60 and 90° C. with an optimum at 80° C. Similarly the study of activity at different pH revealed that enzyme optimal pH were comprised between 7 and 8.5 with an optimum at 7.5 (FIG. 4B).

### Effect of Ions Metallic on the Enzyme Activity

The study of the effect of the metal ions on the activity of the L-AI US100 was undertaken on the enzyme purified according to Example 3 without addition of metal ions in the culture and during purification. Moreover, the purified enzyme was dialysed against 0.1 M MOPS (pH 7.5) containing 10 mm EDTA during 48 hours. The determination of the effect of different metal ions (Mn²⁺, Co²⁺, Ca²⁺, Mg2+, Ni2+, Cu²⁺ and Fe²⁺) were tested by addition of these divalent ions at a final concentration of 0.1 mM in the reactional medium. The obtained results indicated in the Table 1 demonstrated that, at 65° C., these metallic ions do not have any effect on the activity of the enzyme, whereas at 80° C. only the Mn²⁺ ions and Co²⁺ increases apparently the activity of the L-AI US100 until reaching 140% (Table 1). These finding proved that these ions are implicated on the stabilisation of the protein.

### C—Co²⁺ and Mn²⁺ Effect of on the Thermostability

The study of the concentration effect of Mn²⁺ and Co²⁺ on the L-AI US100 thermostability determined at 75° C. proved that the optimal concentration were 1 mM and 0.2 mM respectively (FIG. 5).

The thermal stability of the L-AI US100 was studied after incubation of the enzyme during 30, 60, 90 and 120 min at 65, 70, 75 and 80° C. in presence and absence of metallic ions. In total absence of these ions, the L-AI US100 completely preserves its activity at 65° C., whereas it has a half-life of 10, 60 and 120 min with 80, 75 and 70° C. respectively. In the presence of 1 mM Mn²⁺ and 0.2 mM Co2+, the L-AI US100 retains more than 90% of its initial activity at 70° C. after 120 min of incubation. Moreover, the half-life time reaches 20 and 115 min at 80 and 75° C. respectively. The study of the effect of each ion on stability gives similar results. Consequently, the study of thermostability showed that the L-AI US100 has a feeble requirement for metallic ions for its thermostability comparing to others reported LAIs. In fact the L-AI US100 requires 1 mM Mn²⁺ and 0.2 mM Co²⁺ (Table 2) while the other L-AIs reported requires a higher concentration, such 1 mM Co²⁺ and 5 mM Mn²⁺ required by the L-AI derived from Thermotoga maritima (Table 2) (Kim et al, Biotechnol Lett. June 2003; 25(12) : 963-7; Lee et al, Appl Environ Microbiol. March 2004; 70(3): 1397-404 et Kim et al, FEMS Microbiol Lett. Jun. 18, 2002; 212 (1): 121-6).

### D—Determination of Kinetic Parameters:

To determine the kinetic parameters of the L-AI US100 for L-arabinose and D-galactose, the representations of Lineweaver-Burck were carried out (FIG. 6). According to these representations, we deduce that the L-AI US100 has a km values evaluated to 28.57 mM for L-arabinose and 52.63 mM for D-galactose. Furthermore, the values of Vmax were 40 U/mg and 8.7 U/mg for L-arabinose and D-galactose respectively. Hence the catalytic effectiveness (kcat/Km) of this enzyme for L-arabinose is 71.4 mm⁻¹.min⁻¹ and 8.46 mm⁻¹.min⁻¹ for L-arabinose and D-galactose respectively, which is the highest one after that of Thermotoga maritima.

### Example 4

### Conversion of D-Galactose into D-Tagatose at Different Temperatures

In this example are given data, for indication and not for restriction, concerning the conversion of D-galactose into D-tagatose at different temperatures using the L-AI US100.

Using the L-AI US100, subject of this invention, the conversion rate of D-galactose into D-tagatose was studied at various temperatures and after regular period of time. The reactional medium is composed of 5 mM of D-galactose, 100 mm MOPS (pH 7.5) and the pure enzyme at a final concentration of 3 mg/ml. The D-tagatose produced is measured by the Cysteine-carbazole method (as it was reported in Example 1). In presence of 1 mm Mn²⁺ and 0.2 mM Co²⁺ the maximal D-galactose bioconversion into D-tagatose reaches 48% after 7 h incubation at 70° C. (FIG. 7).

### CITED REFERENCES

1—Kim J W, Kim Y W, Roh H J, Kim H Y, Cha J H, Park K H, Park C S. Production of tagatose by a recombinant thermostable L-arabinose isomerase from Thermus sp.IM6501.Biotechnol Lett. June 2003; 25(12): 963-7.
2—Lee D W, Jang H J, Choe E A, Kim B C, Lee S J, Kim S B, Hong Y H, Pyun Y R. Characterization of a thermostable L-arabinose (D-galactose) isomerase from the hyperthermophilic eubacterium Thermotoga maritima. Appl Environ Microbiol. March 2004; 70(3): 1397-404.
3—Kim B C, Lee Y H, Lee H S, Lee D W, Choe E A, Pyun Y R. Cloning, expression and characterization of L-arabinose isomerase from Thermotoga neapolitana: bioconversion of D-galactose to D-tagatose using the enzyme. FEMS Microbiol Lett. Jun. 18, 2002; 212(1): 121-6.
4—Dische, Z., and Borenfreund, E. A New Spectrophotometric Method for the Detection and Determination of Keto Sugars and Trioses. J. Biol. Chem., 192:583-587, 1951.
5—Mamdouh Ben Ali, Monia Mezghani, and Samir Bejar. A thermostable □-amylase producing maltohexaose from a new isolated bacillus sp. US100: study of activity and molecular cloning of the corresponding gene. Enz. Microb. Technol. 24: 584-589, 1999.
6—Mamdouh Ben Ali, Monia Mezghani, lotfi Mallouli, Sonda Mhiri, Radhouane Ellouze et Samir Bejar. Une nouvelle activité amylase thermostable et productrice de maltohexaose: caractérisation de l'activité, de la protéine et du gène correspondant. Brevet d'invention No: 17236, INNORPI TUNISIE, 2001.

TABLE 1

| Metallic ions | Relative activity (%) | Relative activity (%) |
|---|---|---|
| (0.1 mM) | at 65° C. | at 80° C. |
| None ^{(A)} | 100 | 100 |
| Cucl₂ | 25 | 13.8 |
| Cacl₂ | 100 | 96 |
| Nicl₂ | 100 | 100 |
| Zncl₂ | 95 | 85 |
| Mgcl₂ | 100 | 93 |
| FeSO₄ | 100 | 95 |
| Mncl₂ | 100 | 124 |
| Cocl₂ | 100 | 115 |
| Cocl₂ + Mncl₂ ^{(B)} | 100 | 140 |

^{A}100% represents the activity measured in absence of metallic ions
^{B}The ions Co²⁺ and Mn²⁺ are added at final concentration of 0.1 mM.

TABLE 2

| | | | | Km/kcat (mM · | |
|---|---|---|---|---|---|
| | Optimal | | Metallic ions | min⁻¹) | |
| | temperature | | requirement | L-arabinose/D- | Conversion |
| | (° C.) | pH | (mM) | galactose | rate (%) |
| B. stea. US100 | 80 | 7.5-8 | 0.2 Co²⁺/1 Mn²⁺ | 71.7/8.46 | 48%/7h |
| Thermus sp. | 60 | 8 | 5 Mn²⁺ | nd | 54%/3j |
| Ther. maitima | 80 | 7-7.5 | 1 Co²⁺/5 Mn²⁺ | 74.8/8.5 | 56%/6h |
| Ther. neapolitana | 85 | 7.5 | 1 Co²⁺/1 Mn²⁺ | 51.8/3.24 | 68%/20h |

1. A polynucleotide, encoding a polypeptide having an L-arabinose isomerase activity and comprising the a sequence of SEQ ID NO. 2.

2. A polypeptide, encoded by nucleotide sequence SEQ ID NO. 1, and having an L-arabinose isomerase activity permitting the bioconversion of the D-galactose into D-tagatose.

3. A polypeptide of claim 2, exhibiting minimum dependence on metal ions for its activity and for thermostability.

4. An expression vector harbouring the polynucleotide of claim 1.

5. Recombinant strains able to produce the polypeptide of claim 2.

6. A method of production of L-arabinose isomerase, the method comprising growing a culture of a strain harbouring the expression vector of claim 4 in a culture medium.

7. A method of production of D-tagatose comprising the bioconversion of a D-galactose solution into D-tagatose solution with the polypeptide according to claim 2.

8. A method in accordance with claim 7, where the bioconversion is done with a pH between 6.5 and 8.5 and a temperature between 60 and 90° C.

9. An L-arabinose isomerase, L-AI US 100, including the polypeptide of claim 2.

10. The L-arabinose isomerase of claim 9 in free form.

11. The L-arabinose isomerase of claim 9, wherein the L-arabinose is in the form of an immobilized enzyme.

12. The polynucleotide of claim 1, wherein the polynucleotide is isolated from Bacillus stearothermophilus (strain US 100).


SEQUENCE LISTING (2 sequences)

SEQ ID NO: 1 - DNA, 1491 bp, Bacillus stearothermophilus
  araA US100 gene
atgctgtcattacgtccttatgaattttggtttgtgacaggaagtcagcacttgtacggagaagaagcgttaaaacaagtcgaagaacattcaagaatcatggtcaatgaatggaatcgcgattcggtgtttccgttcccattcgttttcaaatcagtcgtgacgacgccagaggaaatccggcgcgtttgccttgaggcgaatgcgagcgaacaatgcgctggggtcatcacttggatgcatacattctcgccagcgaaaatgtggattggcggccttttggagttgagaaaaccgttattgcatcttcacacccagtttaaccgtgatattccgtgggacagcatcgatatggactttatgaacttaaaccaatcggctcacggtgaccgggaatacggatttatcggcgcgagaatgggcgtggcccggaaagtggtggtcgggcactgggaagacccagaagtccgcgagcggctggcgaaatggatgcggacggctgtcgcgtttgcggaaagccgcaacctaaaagtggctcgttttggcgacaacatgcgtgaagtggctgtgacggaaggggacaaagtcggagcgcaaattcaattcggctggtcggtcaacggctatggcatcggggatttggtgcaatacatccgcgatgtttctgaacaaaaagtgaacgagttgctcgatgaatacgaggagctgtacgacattgtacccgccggccgccaagaagggcccgttcgcgaatcaattcgtgaacaggcgcggattgaactcgggctgaaagcctttttgcaagacgggaacttcactgcctttacgacgacgtttgaagatttgcacggcatgaagcaacttccaggactagcggttcaacggcttatggcagagggatatggatttggcggcgaaggcgactggaaaacggctgctctcgttcggttgatgaaagtcatggcggatggcaaaggaacatcgttcatggaagactacacgtaccactttgagctgggcaacgaactgattcttggcgcgcatatgctcgaagtatgcccgacgattgccgcaacccggccgcgcatcgaagtccatccgctttcgatcggtggaaaagaagatccagcccgtctcgtgtttgacggcggcgagggtgctgcggtcaatgcttcgttgattgacttaggacaccgtttccgtctcattgtcaatgaagtcgatgcagtgaaaccagaacatgagatgccgaaactgcctgttgcgcgcattctatggaaaccgcgcccgtcactccgcgactcggccgaagcatggattttagccggcggagcgcatcacacatgcttctcgtttgcggtcacggctgaacagctgcaagactttgcggaaatggcgggcattgaatgcgtcgtgatcaatgaacatacgtccgtctcctcattcaagaacgaactaagatggaatgaagtattctggcgggggcggtaa

SEQ ID NO: 2 - protein, 496 aa, Bacillus stearothermophilus
  L-A1 US100 Peptide encoded by araA US100 gene
MLSLRPYEFWFVTGSQHLYGEEALKQVEEHSRIMVNEWNRDSVFPFPFVFKSVVTTPEEIRRVCLEANASEQCAGVITWMHTFSPAKMWIGGLLELRKPLLHLHTQFNRDIPWDSIDMDFMNLNQSAHGDREYGFIGARMGVARKVVVGHWEDPEVRERLAKWMRTAVAFAESRNLKVARFGDNMREVAVTEGDKVGAQIQFGWSVNGYGIGDLVQYIRDVSEQKVNELLDEYEELYDIVPAGRQEGPVRESIREQARIELGLKAFLQDGNFTAFTTTFEDLHGMKQLPGLAVQRLMAEGYGFGGEGDWKTAALVRLMKVMADGKGTSFMEDYTYHFELGNELILGAHMLEVCPTIAATRPRIEVHPLSIGGKEDPARLVFDGGEGAAVNASLIDLGHRFRLIVNEVDAVKPEHEMPKLPVARILWKPRPSLRDSAEAWILAGGAHHTCFSFAVTAEQLQDFAEMAGIECVVINEHTSVSSFKNELRWNEVFWRGR